(19) United States
(12) Patent Application Publication
(10) Pub. No.: US 2021/0000852 A1
(43) Pub. Date: Jan. 7, 2021

(54) POLYDATIN PACLITAXEL COMPOSITION, AND APPLICATION IN PREPARING MEDICAMENT FOR PREVENTING AND TREATING GASTRIC CANCER

(71) Applicant: Tianjin Medical University Cancer Institute and Hospital, Tianjin (CN)

(72) Inventors: Liren LIU, Tianjin (CN); Boyu PAN, Tianjin (CN)

(21) Appl. No.: 16/975,332

(22) PCT Filed: Feb. 28, 2019
(86) PCT No.: PCT/CN2019/076430

§ 371 Date: Aug. 24, 2020

(30) Foreign Application Priority Data
Mar. 2, 2018 (CN) 201810175965.2

(51) Int. Cl. A61K 31/7034; A61K 31/337; A61P 35/00

(52) U.S. Cl. CPC A61K 31/7034; A61P 35/00; A61K 31/337

(57) ABSTRACT

The invention discloses a Polydatin Paclitaxel composition comprising Polydatin and Paclitaxel, and application in preparing a medicament for preventing and treating gastric cancer. Biologic function experiments in cells of the invention mainly demonstrate that Polydatin and Paclitaxel combined at a mass ratio of (22.65-33.77):1 may synergistically inhibit the activity of human gastric cancer cells; and achieve treatment effects of reducing toxicity and enhancing efficacy. Therefore, Polydatin and Paclitaxel may be combined to form a composition as an active component in preparing a medicament for preventing and treating gastric cancer.

This is the United States National Stage of Patent Cooperation Treaty Application No. PCT/CN2019/076430 filed Feb. 28, 2019, which claims priority to Chinese Application No. 201810175965.2 filed Mar. 2, 2018, the disclosures of which are incorporated herein by reference in their entireties.

### TECHNICAL FIELD

The invention belongs to the field of medical use, and relates to application of a Polydatin Paclitaxel composition in preparing a medicament for preventing and treating gastric cancer.

### BACKGROUND

Currently, malignant tumors pose a serious threat to human health and life. The incidence rate of malignant tumors continues to increase largely because of the aging and growth of the world population alongside an increasing adoption of cancer-causing behaviors^{[1]}. Gastric cancer is a common malignancy in the world. According to the statistical data of International Agency for Research on Cancer (IARC), there are about 95.1 million new cases and about 72.3 million deaths due to gastric cancer in 2012, ranking fifth place for malignancy incidence and third place for mortality. With occult onset and less early specific symptoms, majority of gastric cancer patients have progressed to an advanced stage at the time of first diagnosis. However, currently, some traditional chemotherapy regimens for patients with gastric cancer clinically have poor survival and prognosis for the patients with advanced-stage cancer; and targeted drug therapies are usually not suitable for every patient. Therefore, it is of great significance to continue in-depth exploration and screening of novel clinical anti-cancer drugs for the discovery of new methods for the prevention and treatment of gastric cancer, and to seek breakthroughs in broad-spectrum treatment of gastric cancer.

The active ingredients extracted from natural plants play an important role in the field of chemotherapy for malignant tumors. Of the 155 small molecule anti-neoplastic drugs developed from 1940 to 2006, it was reported that, nearly 47% actually are either natural products or directly derived therefrom^{[4]}, including Paclitaxel derived from Taxus chinensis. Paclitaxel, a complex tetracyclic diterpenoid compound, was first extracted from the stem bark of Taxus brevifolia Nutt. by Wani etc. in 1971, when its chemical structure was determined^{[5]}. At present, Paclitaxel has been widely used for treating breast cancer, ovarian cancer, lung cancer, head & neck cancer and gastrointestinal cancer, albeit with some toxic and side effects, such as alopecia, myelosuppression, facial flushing and muscle joint soreness.

Polydatin (PD) is the fourth monomer extracted from the dried rhizome of Polygonum Cuspidatum in the Polygonaceae family, also known as crystal No. 4 of Polygonum cuspidatum or resveratrol glycoside, which is widely found in grape and wine. Recent studies have shown that PD has significant effects on a variety of biological processes, such as anti-platelet aggregation/antithrombotic formation, strengthening contraction and relaxation functions of myocardial cells, and improving microcirculation of important tissues and organs after shock^{[6-12]}; in addition, PD has the effects of relieving chronic pain associated with endometriosis and ameliorating colitis^{[13,14]}. Moreover, as a derivative of resveratrol, PD has been shown to have a good effect on the prevention and treatment of various malignancies^{[15-18]}. However, the combination of PD and Paclitaxel in the prevention and treatment of gastric cancer has not been reported. Therefore, it has become one of the hotspots in the research field of cancer therapy to screen novel anti-neoplastic drugs from natural plants, determine their therapeutic effects as well as the mechanisms involving toxicity and efficacy.

### SUMMARY

The invention aims to overcome the defects in the prior art, and provides application of a Polydatin Paclitaxel composition in preparing a medicament for preventing and treating gastric cancer.

The technical scheme of the invention is summarized as follows:

A Polydatin Paclitaxel composition comprises Polydatin and Paclitaxel.

A mass ratio of the Polydatin to the Paclitaxel is preferably (22.65-33.77):1.

The Polydatin has a molecular formula C₂₀H₂₂O₈ as follows:

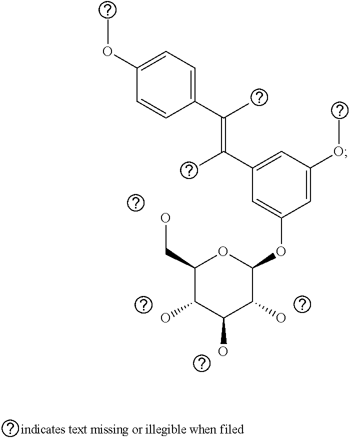 [SMILES: COc1ccc(/C(C)=C(\C)c2cc(OC)cc(O[C@@H]3O[C@H](CO)[C@@H](O)[C@H](O)[C@H]3O)c2)cc1]

The Paclitaxel has a molecular formula C₄₇H₅₁NO₁₄ as follows:

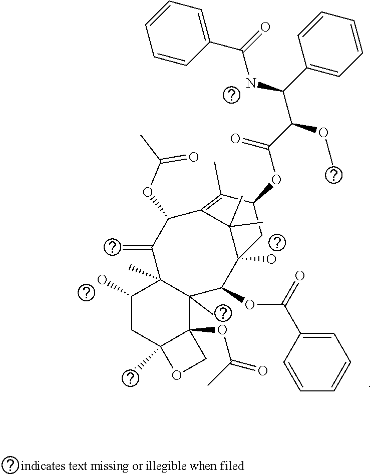 [SMILES: C=C1[C@H](OC(C)=O)C2=C(C)[C@@H](OC(=O)[C@H](OC)[C@@H](NC(=O)c3ccccc3)c3ccccc3)C[C@@](O)([C@@H](OC(=O)c3ccccc3)C3(C)[C@@]1(C)[C@@H](O)C[C@]1(C)OC[C@@]31OC(C)=O)C2(C)C]

The invention relates to application of a Polydatin Paclitaxel composition in preparing a medicament for preventing and treating gastric cancer.

The gastric cancer may be intestinal type, diffuse type or mixed type.

Functional experiments in cells of the invention mainly demonstrate that Polydatin and Paclitaxel combined at a mass ratio of (22.65-33.77):1 may synergistically inhibit the activity of human gastric cancer cells; and achieve treatment effects of reducing toxicity and enhancing efficacy. Therefore, Polydatin and Paclitaxel may be combined to form a composition as the active component in preparing a medicament for preventing and treating gastric cancer.

### BRIEF DESCRIPTION OF THE DRAWING

FIG. 1 shows observations of gastric cancer cells SGC-7901 treated with Polydatin at different concentration gradients (0 μg/ml; 25 μg/ml; 50 μg/ml; 100 μg/ml; 200 μg/ml; 400 μg/ml) for 24 h.

FIG. 2 shows observations of gastric cancer cells AGS treated with Polydatin at different concentration gradients (0 μg/ml; 25 μg/ml; 50 μg/ml; 100 μg/ml; 200 μg/ml; 400 μg/ml) for 24 h.

FIG. 3 shows observations of gastric cancer cells SGC-7901 treated with Paclitaxel at different concentration gradients (0 μg/ml; 2.5 μg/ml; 5 μg/ml; 10 μg/ml; 20 μg/ml; 30 μg/ml) for 24 h.

FIG. 4 shows observations of gastric cancer cells AGS treated with Paclitaxel at different concentration gradients (0μg/ml; 2.5μg/ml; 5 μg/ml; 10μg/ml; 20μg/ml; 30μg/ml) for 24 h.

FIG. 5 shows observations of synergistic effect of Polydatin and Paclitaxel on human gastric cancer cells SGC-7901 treated with a Polydatin Paclitaxel composition at different mass ratios for 24 h. Note: FIG. 5(a) A: A control group; B: Paclitaxel IC₅₀ group; C: PD IC₅₀ group; D: Paclitaxel IC₅₀+PD IC₅₀ group. FIG. 5(b) A: A control group; B: Paclitaxel 0.5 IC₅₀ group; C: PD IC₅₀ group; D: Paclitaxel 0.5 IC₅₀+PD IC₅₀ group. FIG. 5(c) A: A control group; B: Paclitaxel 0.25 IC₅₀ group; C: PD IC₅₀ group; D: Paclitaxel 0.25 IC₅₀+PD IC₅₀ group. FIG. 5(d) A: A control group; B: Paclitaxel 0.1 IC₅₀ group; C: PD IC₅₀ group; D: Paclitaxel 0.1 IC₅₀+PD IC₅₀ group.

FIG. 6 shows observations of synergistic effect of Polydatin and Paclitaxel on human gastric cancer cells AGS treated with a Polydatin Paclitaxel composition at different mass ratios for 24 h. Note: FIG. 6(a) A: A control group; B: Paclitaxel IC₅₀ group; C: PD IC₅₀ group; D: Paclitaxel IC₅₀+PD IC₅₀ group. FIG. 6(b) A: A control group; B: Paclitaxel 0.5 IC₅₀ group; C: PD IC₅₀ group; D: Paclitaxel 0.5 IC₅₀+PD IC₅₀ group. FIG. 6(c) A: A control group; B: Paclitaxel 0.25 IC₅₀ group; C: PD IC₅₀ group; D: Paclitaxel 0.25 IC₅₀+PD IC₅₀ group.

FIG. 7 shows observations of toxicity-reducing and efficacy-enhancing effects of Polydatin and Paclitaxel on human gastric cancer cells SGC-7901 treated with a Polydatin Paclitaxel composition at different mass ratios for 24 h. Note: FIG. 7(a) A: A control group; B: Paclitaxel IC₅₀ group; C: Paclitaxel 0.5 IC₅₀ group; d: Paclitaxel 0.5 IC₅₀+PD IC₅₀ group. FIG. 7(b) A: A control group; B: Paclitaxel IC₅₀ group; C: Paclitaxel 0.25 IC₅₀ group; d: Paclitaxel 0.25 IC₅₀+PD IC₅₀ group. FIG. 7(c) A: A control group; B: Paclitaxel IC₅₀ group; C: Paclitaxel 0.1 IC₅₀ group; d: Paclitaxel 0.1 IC₅₀+PD IC₅₀ group.

FIG. 8 shows observations of toxicity-reducing and efficacy-enhancing effects of Polydatin and Paclitaxel on human gastric cancer cells AGS treated with a Polydatin Paclitaxel composition at different mass ratios for 24 h. Note: FIG. 8(a) A: A control group; B: Paclitaxel IC₅₀ group; C: Paclitaxel 0.5 IC₅₀ group; d: Paclitaxel 0.5 IC₅₀+PD IC₅₀ group. FIG. 8(b) A: A control group; B: Paclitaxel IC₅₀ group; C: Paclitaxel 0.25 IC₅₀ group; d: Paclitaxel 0.25 IC₅₀+PD IC₅₀ group.

### DETAILED DESCRIPTION

Human gastric cancer cell line SGC-7901 was purchased from Shanghai Genechem Co., Ltd. and human gastric cancer cell line AGS was purchased from Cell Bank of American Type Culture Collection (ATCC) (ATCC No. CRL-1739).

The invention will be further described below with reference to specific examples.

### EXAMPLE 1

Polydatin has a molecular formula C₂₀H₂₂O₈ as follows:

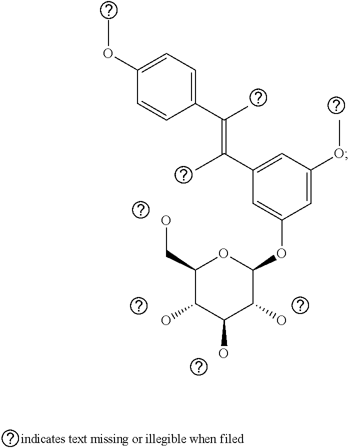 [SMILES: COc1ccc(/C(C)=C(\C)c2cc(OC)cc(O[C@@H]3O[C@H](CO)[C@@H](O)[C@H](O)[C@H]3O)c2)cc1]

Paclitaxel has a molecular formula C₄₇H₅₁NO₁₄ as follows:

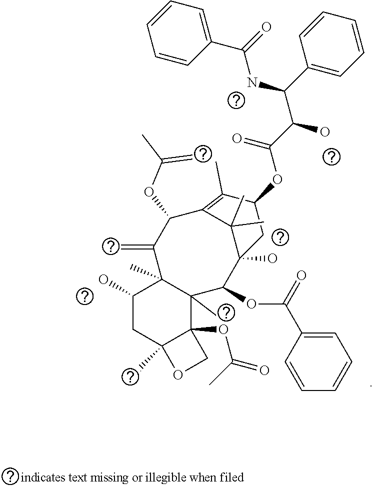 [SMILES: C=C(C)O[C@H]1C(=C)[C@]2(C)[C@@H](O)C[C@]3(C)OC[C@@]3(OC(C)=O)C2(C)[C@H](OC(=O)c2ccccc2)[C@]2(O)C[C@H](OC(=O)[C@H](O)[C@@H](NC(=O)c3ccccc3)c3ccccc3)C(C)=C1C2(C)C]

With a Polydatin Paclitaxel composition as an active component, liquid injections of various specifications may be prepared according to conventional methods by adding pharmaceutically acceptable adjuvants.

The Polydatin Paclitaxel composition may be administrated by various routes such as injection administration, intracavitary administration and the like.

### EXAMPLE 2

Disclosed is application of a Polydatin Paclitaxel composition in preparing a medicament for preventing and treating a gastric malignant tumor, where the composition comprises Polydatin and Paclitaxel at a mass ratio of 28.21:1.

### EXAMPLE 3

Disclosed is application of a Polydatin Paclitaxel composition in preparing a medicament for preventing and treating a gastric malignant tumor, where the composition comprises Polydatin and Paclitaxel at a mass ratio of 22.65:1.

### EXAMPLE 4

Disclosed is application of a Polydatin Paclitaxel composition in preparing a medicament for preventing and treating a gastric malignant tumor, where the composition comprises Polydatin and Paclitaxel at a mass ratio of 33.77:1.

### EXAMPLE 5

Polydatin inhibited the activity of various human gastric cancer cells.

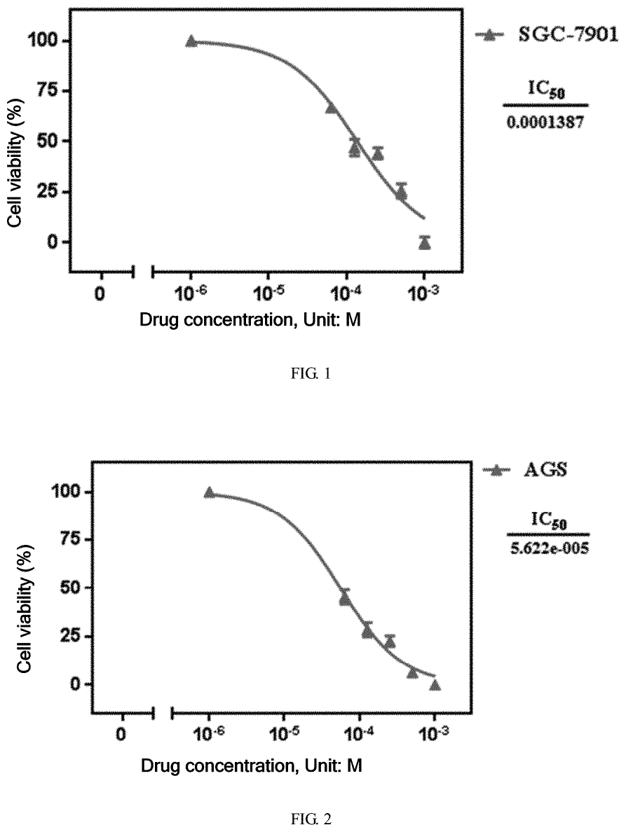

Human gastric cancer cell lines SGC-7901 and AGS were selected for the experiments, and it was found that: PD could effectively inhibit the growth and proliferation activity of human gastric cancer cells through the CCK-8 method (FIG. 1, FIG. 2); PD could inhibit the activity of both gastric cancer cells in a concentration-dependent manner for 24 h; the IC₅₀ of SGC-7901 and AGS cells treated with PD for 24 h were as follows: 1.387×10⁻⁴M (124.0μg/mL) and 5.622×10⁻⁵M (53.5 μg/mL).

### EXAMPLE 6

Paclitaxel inhibited the activity of various human gastric cancer cells.

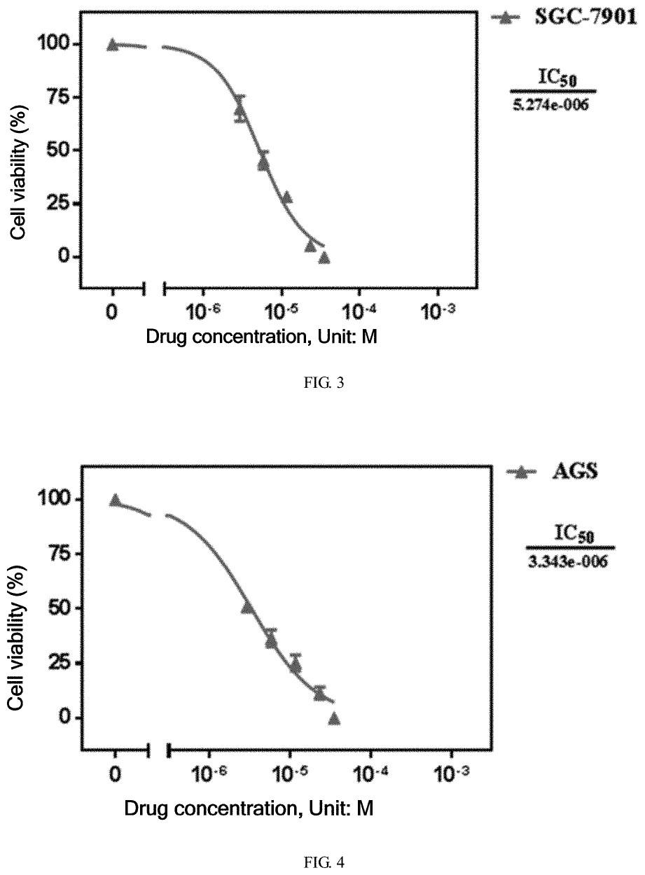

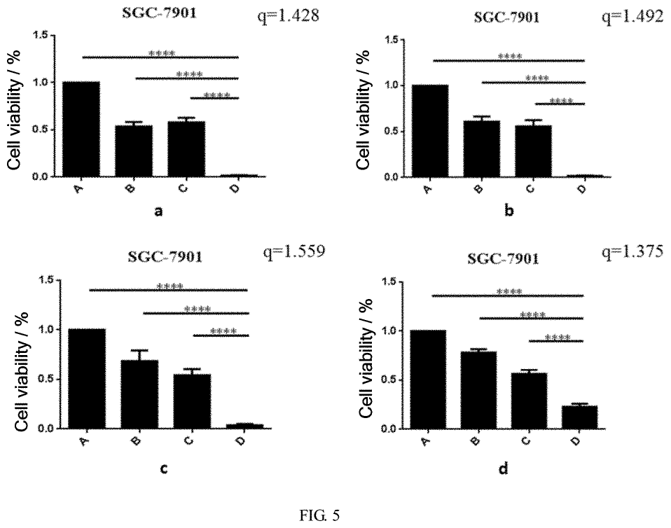

Human gastric cancer cell lines SGC-7901 and AGS were selected for the experiments, and it was found that: Paclitaxel could effectively inhibit the growth and proliferation activity of human gastric cancer cells through the CCK-8 method (FIG. 3, FIG. 4); Paclitaxel could inhibit the activity of both gastric cancer cells in a concentration-dependent manner for 24 h. The IC₅₀ of SGC-7901 and AGS cells treated with Paclitaxel for 24 h were as follows: 5.274×10⁻⁶ M (5.5 μg/mL) and 3.343×10⁻⁶M (6.3 μg/mL).

### EXAMPLE 7

The composition of the invention synergistically inhibited the activity of human gastric cancer cells.

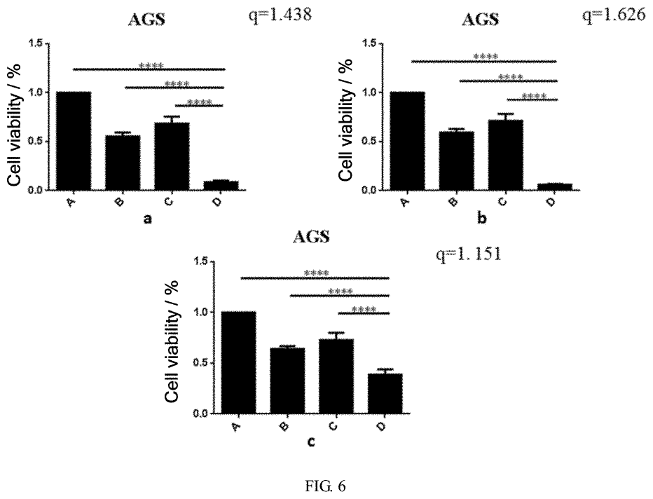

Human gastric cancer cell lines SGC-7901 and AGS were selected for the experiments, and it was found that: the composition of the invention had a significant inhibitory effect on the growth and proliferation activity of human gastric cancer cells compared with the respective single-agent effect through the CCK-8 method (FIG. 5, FIG. 6); the composition of PD and Paclitaxel could significantly inhibit the growth activity of both gastric cancer cells for 24 h. In FIG. 5, the mass ration of composition is as follows: (a) m_{PD}: m_{Paclitaxel}=22.65:1 (Example 3); (b) m_{PD}: m_{Paclitaxel}=45.29:1; (c) m_{PD}: m_{Paclitaxel}=90.59:1; (d) m_{PD}: m_{Paclitaxel}=226.48:1. In FIG. 6, the mass ration of composition is as follows: (a) m_{PD}: m_{Paclitaxel}=8.44:1; (b) m_{PD}: m_{Paclitaxel}=16.88:1; (c) m_{PD}: m_{Paclitaxel}=33.77:1 (Example 4).

### EXAMPLE 8

The composition provided by the invention has the treatment effects of reducing toxicity and enhancing efficacy.

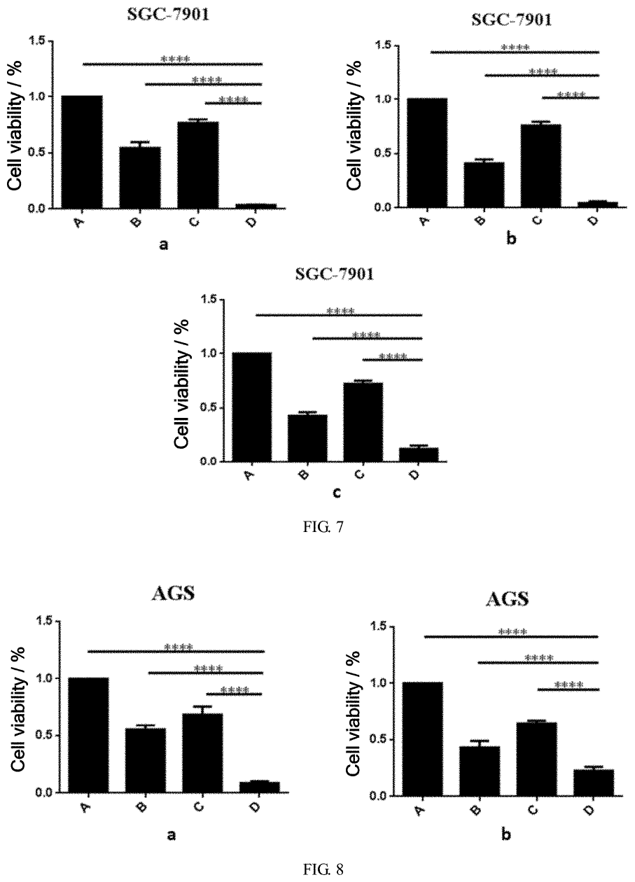

We selected human gastric cancer cell lines SGC-7901 and AGS for experiments, and found that: PD and Paclitaxel composition had a significant inhibitory effect on the growth and proliferation activity of human gastric cancer cells compared with Paclitaxel single-agent through the CCK-8 method (FIG. 7, FIG. 8); the composition of PD and Paclitaxel could significantly inhibit the growth activity of both gastric cancer cells for 24 h, and the effect was better than that of respective Paclitaxel IC₅₀ group. In FIG. 7, the mass ration of composition is as follows: (a) m_{PD}: m_{Pactitaxel}=45.29:1; (b) m_{PD}: m_{Paclitaxel}=90.59:1; (c) m_{PD}: m_{Paclitaxel}=226.48:1. In FIG. 8, the mass ration of composition is as follows: (a) m_{PD}: m_{Paclitaxel}=16.88:1; (b) m_{PD}: m_{Paclitaxel}=33.77:1.

The results demonstrated that Polydatin and Paclitaxel combined at a mass ratio of (22.65-33.77):1 may synergistically inhibit the growth activity of human gastric cancer cells; and achieve treatment effects of reducing toxicity and enhancing efficacy. The Polydatin Paclitaxel composition may be used as an effective component in preparing a medicament for inhibiting the growth of human gastric cancer cells because of the above function.

The tumor is a gastric malignant tumor and comprises intestinal type, diffuse type or mixed type according to Lauren classification.

### MAIN REFERENCES

1. Jemal A, Bray F, Center M M, et al. Global cancer statistics. CA Cancer J Clin. 2011; 61(2):69-90.

2. Newman D J, Cragg G M. Natural products as sources of new drugs over the last 25 years. Nat Prod.2007; 70(3):461-477.

3. Wani M C, Taylor H L, Wall M E, et al. Plant antitumor agents. VI. The isolation and structure of taxol, a novel antileukemic and antitumor agent from Taxus brevifolia. J Am Chem Soc, 1971,93(9):2325.

4. Gao J P, Chen C X, Gu W L, et al. Effects of polydatin on attenuating ventricular remodeling in isoproterenol-induced mouse and pressure-overload rat models. Fitoterapia. 2010; 81(7):953-960.

5. Zhang L P, Ma H J, Bu H M, et al. Polydatin attenuates ischemia/reperfusion-induced apoptosis in myocardium of the rat. Sheng Li Xue Bao. 2009; 61:367-72.

6. Zhang P W, Yu C L, Wang Y Z, et al. Influence of 3,4,5-trihydroxystibene-3-beta-mono-D-glucoside on vascular endothelial epoprostenol and platelet aggregation. Acta Pharmacologica Sinica. 1995; 16:265-268.

7. Wang X, Song R, Chen Y et al. Polydatin-a new mitochondria protector for acute severe hemorrhagic shock treatment. Expert Opin Investig Drugs.2013; 22(2):169-179.

8. Wang X, Song R, Bian H N, et al. Polydatin, a natural polyphenol, protects arterial smooth muscle cells against mitochondrial dysfunction and lysosomal destabilization following hemorrhagic shock. Am J Physiol Regul Integr Comp Physiol.2012; 302(7):R805-814.

9. Cheng Y, Zhang H T, Sun L, et al. Involvement of cell adhesion molecules in polydatin protection of brain tissues from ischemia-reperfusion injury. Brain Res. 2006; 1110(1):193-200.

10. Miao Q, Wang S, Miao S, et al. Cardioprotective effect of polydatin against ischemia/reperfusion injury: roles of protein kinase C and mito K(ATP) activation. Phytomedicine. 2011; 19(1):8-12.

11. Indraccolo U, Barbieri F. Effect of palmitoylethanolamide-polydatin combination on chronic pelvic pain associated with endometriosis: preliminary observations. Eur J Obstet Gynecol Reprod Biol.2010; 150(1):76-79.

142. Yao J, Wang J Y, Liu L, et al. Polydatin ameliorates DSS-induced colitis in mice through inhibition of nuclear factor-kappa B activation. Planta Med.2011; 77(5):421-427.

13. Liu P L, Tsai J R, Charles A L, et al. Resveratrol inhibits human lung adenocarcinoma cell metastasis by suppressing heme oxygenase 1-mediated nuclear factor-kappaB pathway and subsequently downregulating expression of matrix metalloproteinases. Mol Nutr Food Res. 2010; 54(suppl 2): S196-S204.

14. Zhou H B, Chen J J, Wang W X, et al. Anticancer activity of resveratrol on implanted human primary gastric carcinoma cells in nude mice. World J Gastroenterol.2005; 11(2):280-284.

15. Busquets S, Ametller E, Fuster G, et al. Resveratrol, a natural diphenol, reduces metastatic growth in an experimental cancer model. Cancer Lett.2007; 245(1-2):144-148.

16. Kim K H, Back J H, Zhu Y, et al. Resveratrol targets transforming growth factor-β2 signaling to block UV-induced tumor progression. J Invest Dermatol.2011; 131(1):195-202.

1. A Polydatin Paclitaxel composition, wherein the composition comprises Polydatin and Paclitaxel.

2. The composition according to claim 1, wherein a mass ratio of the Polydatin to the Paclitaxel is (22.65-33.77):1.

3. The composition according to claim 1, wherein the Polydatin has a molecular formula C₂₀H₂₂O₈ as follows:

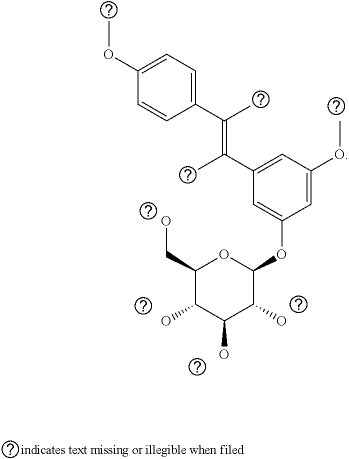 [SMILES: COc1ccc(/C(C)=C(\C)c2cc(OC)cc(O[C@@H]3O[C@H](CO)[C@@H](O)[C@H](O)[C@H]3O)c2)cc1]

4. The composition according to claim 1, wherein the Paclitaxel has a molecular formula C₄₇H₅₁NO₁₄ as follows:

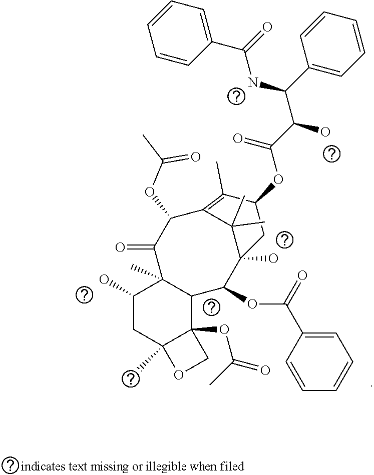 [SMILES: CC(=O)O[C@H]1C(=O)[C@@]2(C)C([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@]1(C)C[C@@H]2O]

5. Application of the Polydatin Paclitaxel composition according to claim 1 in preparing a medicament for preventing and treating gastric cancer.

6. The application according to claim 5, wherein the gastric cancer is intestinal type, diffuse type or mixed type.

7. The composition according to claim 2, wherein the Polydatin has a molecular formula C₂₀H₂₂O₈ as follows:

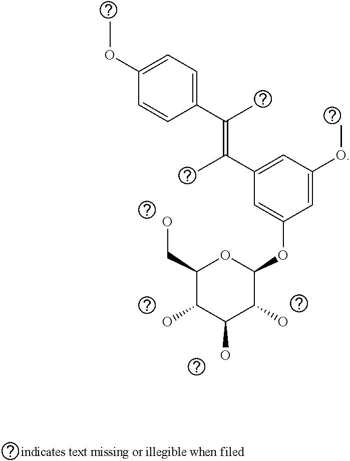 [SMILES: COc1ccc(/C(C)=C(\C)c2cc(OC)cc(O[C@@H]3O[C@H](CO)[C@@H](O)[C@H](O)[C@H]3O)c2)cc1]

8. The composition according to claim 2, wherein the Paclitaxel has a molecular formula C₄₇H₅₁NO₁₄ as follows:

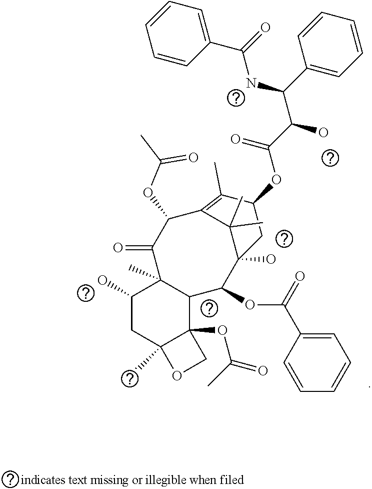 [SMILES: CC(=O)O[C@H]1C(=O)[C@@]2(C)C([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@]1(C)C[C@@H]2O]

9. Application of the Polydatin Paclitaxel composition according to claim 2 in preparing a medicament for preventing and treating gastric cancer.

10. Application of the Polydatin Paclitaxel composition according to claim 3 in preparing a medicament for preventing and treating gastric cancer.

11. Application of the Polydatin Paclitaxel composition according to claim 7 in preparing a medicament for preventing and treating gastric cancer.

12. Application of the Polydatin Paclitaxel composition according to claim 4 in preparing a medicament for preventing and treating gastric cancer.

13. Application of the Polydatin Paclitaxel composition according to claim 8 in preparing a medicament for preventing and treating gastric cancer.

14. The application according to claim 9, wherein the gastric cancer is intestinal type, diffuse type or mixed type.

15. The application according to claim 10, wherein the gastric cancer is intestinal type, diffuse type or mixed type.

16. The application according to claim 11, wherein the gastric cancer is intestinal type, diffuse type or mixed type.

17. The application according to claim 12, wherein the gastric cancer is intestinal type, diffuse type or mixed type.

18. The application according to claim 13, wherein the gastric cancer is intestinal type, diffuse type or mixed type.
